(19) United States
(12) Patent Application Publication
(10) Pub. No.: US 2020/0069156 A1
(43) Pub. Date: Mar. 5, 2020

(54) COUPLING DEVICE FOR A MEDICAL INSTRUMENT

(71) Applicant: WASSENBURG MEDICAL B.V., Dodewaard (NL)

(72) Inventors: Ronald Wassenburg, Dodewaard (NL); Karen Elisa DAMHUIS, Losser (NL); Jan Willem HET LAM, Zwolle (NL); Jeroen Sebastianus Johannes Jozef DESCHEPPER, Wijchen (NL)

(73) Assignee: WASSENBURG MEDICAL B.V., Dodewaard (NL)

(21) Appl. No.: 16/494,144

(22) PCT Filed: Mar. 23, 2018
(86) PCT No.: PCT/NL2018/050181

§ 371 Date: Sep. 13, 2019

(30) Foreign Application Priority Data
Mar. 31, 2017 (NL) 2018624
Mar. 31, 2017 (NL) 2018625

(51) Int. Cl. A61B 1/00; A61B 1/12

(52) U.S. Cl. CPC A61B 1/00128; A61B 1/125

(57) ABSTRACT

The present invention relates to a coupling device for a medical instrument, such as an endoscope, comprising a base part and two cooperating engaging elements connected to the base part, wherein the cooperating engaging elements being movable between a clamping position and a receiving position, wherein each of the cooperating engaging elements being linearly translationally movable between the clamping position and the receiving position, and wherein the translations of the cooperating engaging elements are located on a coinciding line. The present invention further relates to a method for coupling the device according to the present invention with a medical instrument and use of the device according to the present invention, as well as to a kit-of-parts and an insert for use in the device according to the present invention.

The present invention relates to a coupling device for a medical instrument, such as an endoscope. The present invention further relates to a method for coupling the device according to the present invention with a medical instrument and use of the device according to the present invention. The present invention also relates to a kit-of-parts and an insert for use in the device according to the present invention.

Coupling devices for medical instruments, such as channel separators for endoscopes, bronchoscopes and the like are known in the art. Such coupling devices are typically used during the decontamination, i.e. cleaning and disinfecting, of the medical instruments. For example, an endoscope typically includes a plurality of channels which can be configured to direct water, air, and/or any other suitable fluid into a surgical site. In some circumstances, one or more channels in an endoscope can be configured to guide a surgical instrument into the surgical site. In any event, an endoscope includes a control head section having one or more valves, and/or switches, configured to control the flow of fluid through the channels.

Decontamination systems can be used to reprocess previously-used medical devices, such as endoscopes, such that the devices can be used once again. During the decontamination process of, for example, an endoscope, the air and water channels within the endoscope can be evaluated in order to verify that, the channels are unobstructed. Also, a source of fluid can be attached to the channel inlets of the endoscope such that fluid from a fluid source can flow through the channels. In order to avoid a flow of fluid through a channel configured to direct air into a surgical site, coupling devices, such as channel separators are used to separate the channels of a medical device.

In a first aspect of the present invention, the present invention relates to a coupling device for a medical instrument, such as an endoscope, comprising a base part configured for cooperation with at least a part of the medical instrument and two cooperating engaging elements connected to the base part, wherein the cooperating engaging elements being movable between a first position and a second position. In the device according to the present invention, the first position comprises a clamping position, wherein the engaging elements are located at a distance from each other such that the engaging elements clampingly engage at least one part of the medical instrument received by the device, whereas the second position comprises a receiving position, wherein the engaging elements are located at a greater distance from each other relative to the first position such that the device is capable of receiving as well as releasing the at least one part of the medical instrument. The present invention provides a device wherein each of the cooperating engaging elements being linearly translationally movable between the clamping position and the receiving position, and wherein the translations of the cooperating engaging elements are located on a coinciding line. The device of the present invention provides herewith a simple and ergonomic device which facilitates the connection and disconnection between the device of the present invention and at least one part of the medical instrument in a reliable manner. With the device of the present invention the operator, e.g. the user of the coupling device, is able to establish a connection between the device of the present invention and at least one part of the medical instrument by simply exerting a pressure on the device of the present invention, e.g. by squeezing, placing at least a part of the device into the at least one part of the medical instrument, e.g. by sliding the one or more components of the device into the at least one part of the medical instrument, and to establish the connection by releasing the pressure exerted on the device. By releasing the pressure, the engaging elements will translationally move from the receiving position to the clamping position, clampingly connect to the at least one part of the medical instrument.

The present invention thus provides a device wherein, due to the near translational movement of the engaging members the device can be easily positioned by the operator relative to the medical instrument to be connected with the device. Subsequently, without any further effort from the operator, the connection between the device and the medical instrument is established without the need of further changing the position of the device relative to the medical instrument, which facilitates the more ergonomic use of the device, resulting in less user complaints and complications, e.g. injuries like repetitive strain injury (‘RSI’).

The two cooperating engaging elements of the device of the present invention may he substantially conformed, preferably mirrored with respect to one another, in order to establish a reliable connection between the device and the medical instrument wherein the coupling device is repeatedly correctly orientated. Preferably, the two cooperating engaging elements are L-shaped in cross-section, wherein the L-shape of the one engaging element is mirrored with respect to the L-shape of the other engaging element such that, in the clamping position, both engaging elements describe an interrupted U-shape in cross-section. The space between the L-shaped engaging elements in the clamping position, i.e. forming the interrupted ti-shape in cross-section, is designed such that a protruding part of the medical instrument, preferably provided with a flange, is received by the space between the L-shaped engaging elements.

The presence of a flange at the at least one part of the medical instrument prevents any unwanted release of the device in clamping position. Therefore, the two cooperating engaging elements are preferably configured to, in the clamping position, engage under a flange at least one part of the medical instrument received by the device. In a further embodiment, the two cooperating engaging elements describe at least a part of the contours of at least one part of the medical instrument to be received by the device. By describing at least a part of the contours of the at least one part, any unwanted disconnection of the device of the present invention with the medical instrument due to a sliding movement of the device relative to the medical instrument is herewith prevented.

In an embodiment of the present invention, the device of the present invention may be configured such that the engaging elements are under pretension in the receiving position. By bringing the engaging elements are under pretension in the receiving position, the coupling between the device and the medical instrument is facilitate by simply releasing the engaging elements. In other words, the default position of the engaging elements of the device of the present invention is the clamping position. Therefore, the device of the present invention is activated, i.e. bringing the engaging elements under pretension, only in case a medical instrument has to be connected with or disconnected from the coupling device.

In order to facilitate the movement of the engaging elements from the clamping position to the receiving position, each of the two cooperating engaging elements may he connected to a grip or pressure element for translationally moving the engaging element by a user, e.g. the operator, exerting a pressure on the grippers or pressure elements which pressure is directly translated to the engaging elements. By providing a form-retaining assembly of cooperating engaging elements connected to a gripper or pressure element, the pressure exerted on the gripper or pressure element is directly translated to the engaging elements. The device of the present invention therefore provides for a robust and easy to handle coupling device.

Alternatively another device may exert a pressure onto the engaging elements, without the presence of any grippers. Such device may he activated by an intelligent control unit. Even further, another mechanism may be applied to move the engaging elements from the clamping position to the receiving position (and back). Possibly a central activation mechanism may be used wherein an electronic circuit is activated and deactivated by the user. In a further alternative mechanism, the movement of the engaging elements is facilitated using a ballpoint pen-like mechanism for retracting the tip.

In case grips are used, the grips connected to the two cooperating engaging elements are preferably linearly translationally movable between a first grip position and a second grip position, and wherein the translations of the grips are located on a coinciding line. The grips connected to the two cooperating engaging elements in the first grip position are preferably located at a greater distance from each other relative to the second grip position. It is noted that the first grip position preferably corresponds to the clamping position of the engaging elements connected to the grips, and the second grip position preferably corresponds to the receiving position of the engaging elements connected to the grips. In such embodiment of the present invention, a device is provided wherein a pressure exerted on the grips results in a corresponding movement of the engaging elements connected to the grips.

In order to facilitate the ease of use of the grips of the present invention each of the grips connected to the cooperating engaging elements may be provided with at least one fingertip-recess for translationally moving the grip with a fingertip of a user. In a further embodiment, the one grip may be provided with one fingertip-recess for translationally moving the grip with the thumb of a user, whereas the other grip is provided with two fingertip-recesses for translationally moving the grip with the index finger and middle finger of a user.

In order to check the correct coupling between the device and the at least one part of the medical instrument received by the device, each of the grips connected to the cooperating engaging elements may he provided with finger resistances, such as ridges, corrugations or the like.

In a preferred embodiment of the present invention, the device may be configured such that the grips connected to the cooperating engaging elements are under pretension in the second grip position. In other words, the default position for the grips, i.e. the device ‘in rest’, is defined by the grips in the first grip position (corresponding to the engaging elements in the clamping position).

The base part of the device of the present invention may comprise at least one insert, said insert is configured to cooperate with an at least one part of the medical instrument to be received by the device.

In a further embodiment of the present invention, the base part may comprise at least one recess for receiving an insert, said insert is configured to cooperate with an at least one part of the medical instrument to be received by the device. By providing a base part comprising at least one recess for receiving an insert, the interchangeability of inserts is facilitated resulting in a device which is able to he adapted depending on the medical instrument the device has to be coupled with.

Such interchangeability of inserts entails a certain risk, e.g. the inserts may be received by the base part of the device in an incorrect orientation and/or inserts may be located at an incorrect position in case the base part is provided with two or more recesses for receiving inserts. To reduce such risk, the geometry of the at least one recess may be chosen such that the insert can be received only by the base part in a predefined specific orientation. In case the base part comprises at least two recesses for receiving inserts, the geometry of the at least two recesses may be chosen such that they are different with respect to each other.

In a second aspect of the present invention, the present invention relates to a coupling device for a medical instrument, such as an endoscope, comprising a base part configured for cooperation with at least one part of the medical instrument, wherein the device comprises one or more components connected to the base part, such as engaging elements, inserts and the like, wherein the base part comprises a flush connector connectable to a flushing device. In the device of the present invention the flush connector is in direct communication with a channel provided in the base part and the channel comprises at least one opening for flushing one or more components of the device (optionally interchangeably) connected to the base part. By providing a coupling device wherein the base part is provided with a flush connector and an internal channel in direct communication with the flush connector, the base part as such and its components, e.g. the components (interchangeably) connected to the base part and/or the different parts forming the base part can be cleaned, can be disinfected and/or dried using the flush connector in an efficient and reliable manner. The present invention therefore provides a coupling device which can be repeatedly used wherein the formation of contaminations is reduced to an absolute minimum. Unlike rinsing a base part and its components in a cleaning and/or disinfecting medium, the device of the present invention is configured such that the base part and its components can be cleaned and/or disinfected in a targeted manner.

The device of the present invention therefore provides a robust and reliable construction of the base part which can be repeatedly and efficiently flushed after each use. By providing the flush connector of the present invention, the base part and its components can also be dried in a more efficient and targeted manner preventing any microbial growth due to residual liquid after cleaning the base part. Any residual liquid may be removed from the base part by connecting the flush connector to a drying device.

The one or more components of the device connected to the base part may comprise an insert for cooperation with an at least one part of the medical instrument to be received by the device and/or cooperating engaging elements connected to the base part. By providing a base part comprising a channel provided with at least one opening for flushing the one or more components, efficient cleaning and disinfecting of the one or more components is guaranteed. Furthermore, the channel may further facilitate the efficient drying of the one or more components as well.

In a preferred embodiment of the present invention, the channel provided in the base part comprises at least one opening for flushing the engaging elements. In a further embodiment, the base part of the device may comprise one or two recesses for receiving an insert, in such embodiment, the channel may preferably comprise at least one opening debouching into the at least one recess for receiving the insert in order to effectively flush the insert received by the recess of the base part.

The flushing device connectable with the flush connector of the coupling device of the present invention may be configured to flush the device with a flushing medium, such as a flushing liquid and/or air. The flushing liquid may be selected from the group consisting of cleaning liquid, disinfecting liquid, water and combinations thereof. The air may include heated air for the purpose of drying the base part and its components. In a preferred embodiment of the present invention, the flushing device may be part of a cleaning and disinfection machine or drying cabinet for medical instruments, such as endoscopes.

The coupling device of the present invention, i.e. the coupling device of either the first and/or second aspect of the present invention, preferably comprises a channel separator for endoscopes.

In a third aspect of the present invention, the invention relates to a method for coupling the device according to the present invention with a medical instrument. The method according to the present invention comprises the steps of:
a) providing the device according to the first and/or second aspect of the present invention;
b) providing a medical device to be coupled with the medical instrument of step a);
c) bringing the engaging elements of the device provided in step a) in the receiving position;
d) receiving at least one part of the medical instrument provided in step b) by the device provided in step a); and
e) bringing the engaging elements of the device provided in step a) in the clamping position,

wherein, in steps c) and e), the engaging elements linearly translationally move between the receiving position and the clamping position, and wherein the translations of the cooperating engaging elements are located on a coinciding line. Such method facilitates the connection and disconnection between the device of the present invention and at least one part of the medical instrument in a simple and ergonomic manner. As already noted above, the operator is able to establish a connection between the device of the present invention and at least one part of the medical instrument by simply exerting a pressure on the device of the present invention, e.g. by squeezing, placing at least a part of the device into the at least one part of the medical instrument, e.g. by sliding the one or more inserts of the device into the at least one part of the medical instrument, and to establish the connection by releasing the pressure exerted on the device. By releasing the pressure, the engaging elements will translationally move from the receiving position to the clamping position, clampingly connect to the at least one part of the medical instrument.

The present invention thus provides a method wherein, due to the linear translational movement of the engaging members the device can he easily positioned by the operator relative to the medical instrument to be connected with the device. Subsequently, without any further effort from the operator, the connection between the device and the medical instrument is established without the need of further changing the position of the device relative to the medical instrument. The present invention thus provides a simple and robust method for establishing the connection with the device and at least one part of a medical instrument.

The method of the present invention may further comprises the step of f) bringing the engaging elements in the receiving position for releasing the medical instrument coupled to the device. Again it is noted that by simple exerting a pressure on the device of the present invention, e.g. by squeezing the grippers connected to the engaging elements of the device, the operator is able to easily remove the device from the at least one part of the medical instrument the device is connected to.

It is noted that according to the method of the present invention, the receiving position of the engaging elements may be maintained by exerting pressure on the engaging elements, e.g. by exerting pressure on the grippers connected to the engaging elements, wherein the direction of the pressure exerted on the engaging elements corresponds to the direction of the translational movement, of the engaging elements from the clamping position to the receiving position. Such pressure may be exerted on the engaging element by a user, e.g. the operator of the device. However, in an alternative embodiment of the present invention the pressure may be exerted by another device, e.g. initiated or activated by an intelligent control unit.

In a fourth aspect of the present invention, the present invention relates to a kit-of-parts comprising the device according to the first and/or second aspect of the present invention, in particular the device wherein the base part of the device comprises at least one recess for receiving an insert, and a collection of inserts receivable by the device. The collection of inserts is preferably chosen such that the coupling device may be connected to multiple types of medical instruments, e.g. different types of endoscopes wherein each of the endoscopes requires a coupling device comprising specifically designed inserts. By providing a coupling device wherein the inserts can be chosen by the end-user, e.g. the operator of the medical instrument, a device can be configured such that the device is custom-made for the medical instrument the device has to be connected to.

In a fifth aspect of the present invention, the present invention relates to an insert for use in the device according to the first and/or second aspect of the present invention, in particular the device wherein the base part of the device comprises at least one recess for receiving an insert. The present invention provides hereto an insert which is entirely made of plastic. By providing an insert entirely made of plastic, the insert may be disposed directly after use. A single-use insert is thus provided further reducing the chance of using a contaminated not properly cleaned insert.

It is noted that the insert of the present invention differs from the inserts currently available in that the insert is entirely made of plastic material. The inserts currently available on the market are typically made of stainless steel comprising one or more sealing rings made of a different material, such as a plastic material or rubber. By providing an insert made of different materials, the combination results in an additional risk of unwanted contamination of the insert, in particular at the locations the different materials connect to each other. By providing an insert entirely made of a plastic, material, such an additional risk is avoided.

The insert may he made from the same plastic material. Alternatively the properties of the plastic material of the insert may differ for one or more particular surface parts of the insert compared to the properties of the plastic material for the remainder of the surface parts of the insert. In case an insert is provided wherein the properties of the plastic material for certain surface parts deviates from the properties of the plastic material for the remainder of the surface parts, such insert is preferably manufactured using appropriate moulding techniques, such as injection moulding. A preferred moulding technique for manufacturing the inserts of the present invention comprises a two component injection moulding (2K injection moulding) process.

To avoid incorrect usage of the disposable (and easily interchangeable) inserts of the present invention, e.g. by connecting an insert to the base part in a wrong, non-functioning orientation, the geometry of the inserts may be chosen such that this geometry is complementary to the geometry of the at least one recess provided in the base part for receiving the insert. The geometry of an insert may he recess specific, not only to avoid an incorrect oriented connection of the insert with the base part, but also to avoid the incorrect positioning of the insert in case the base part is provided with two or more recesses for receiving inserts.

In a sixth, and final, aspect of the present invention, the present invention relates to the use of the device of the present invention in a method for cleaning, disinfecting and/or drying of a medical instrument, such as an endoscope.

The present invention will be further elucidated on the basis of the non-limitative exemplary embodiments shown in the following figures. Herein shows:

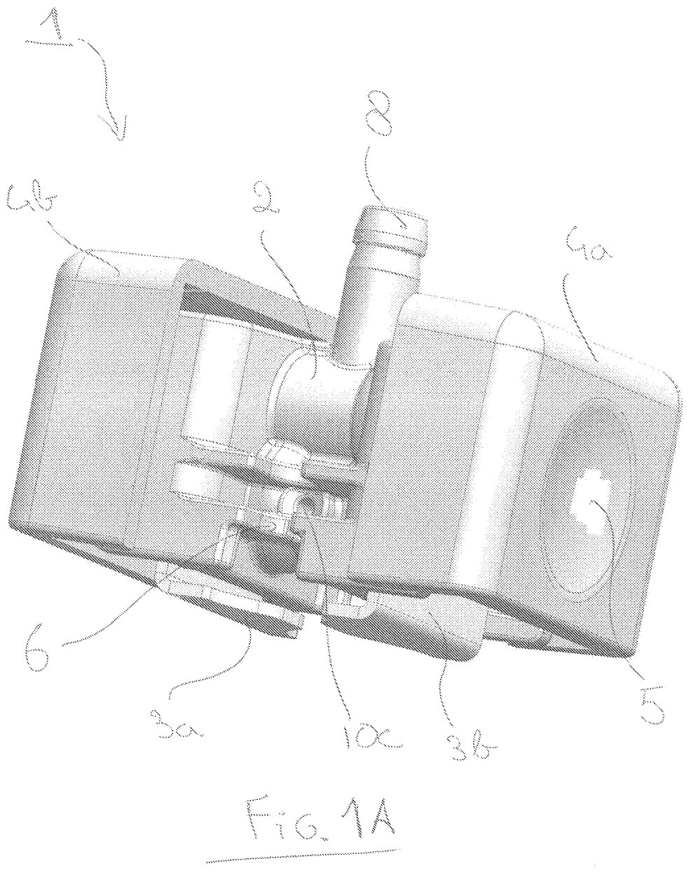

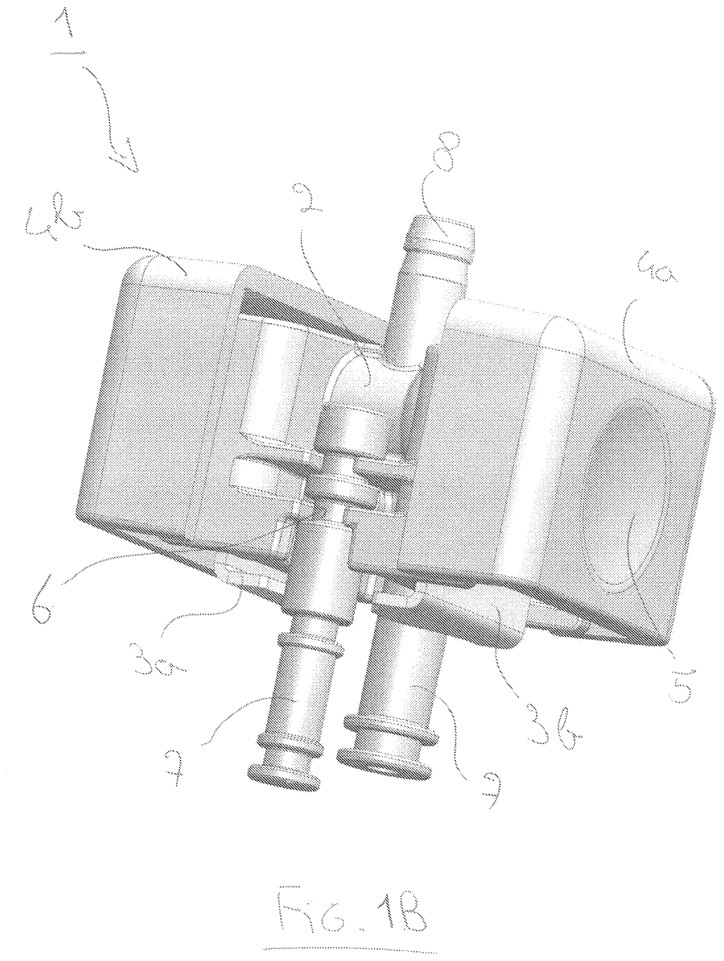

FIG. 1A and 1B a perspective view of the coupling device according to the present invention;

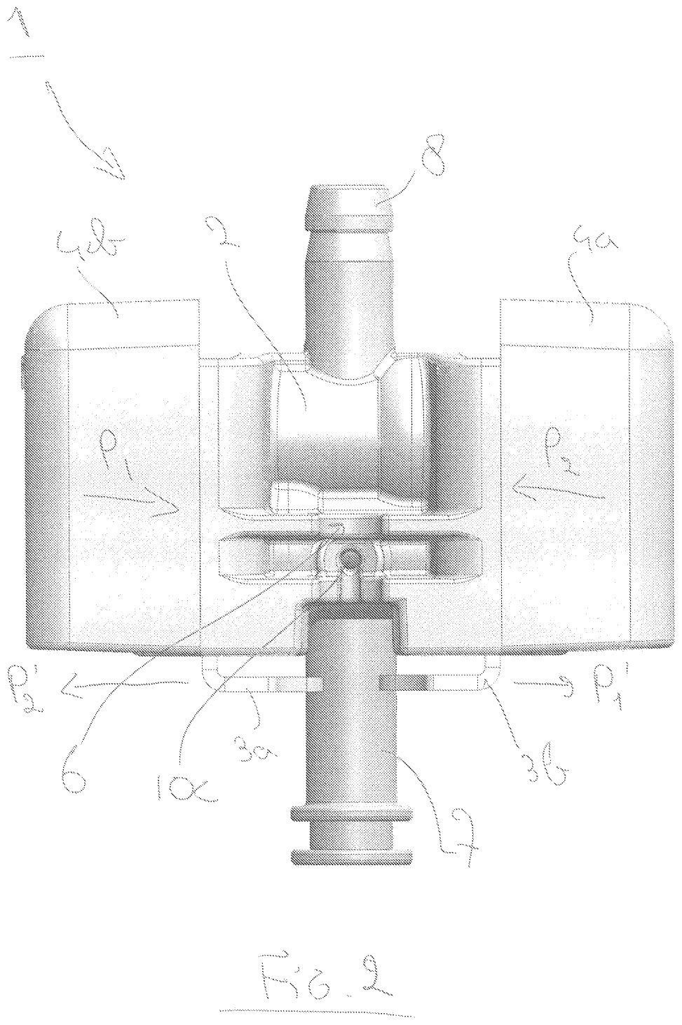

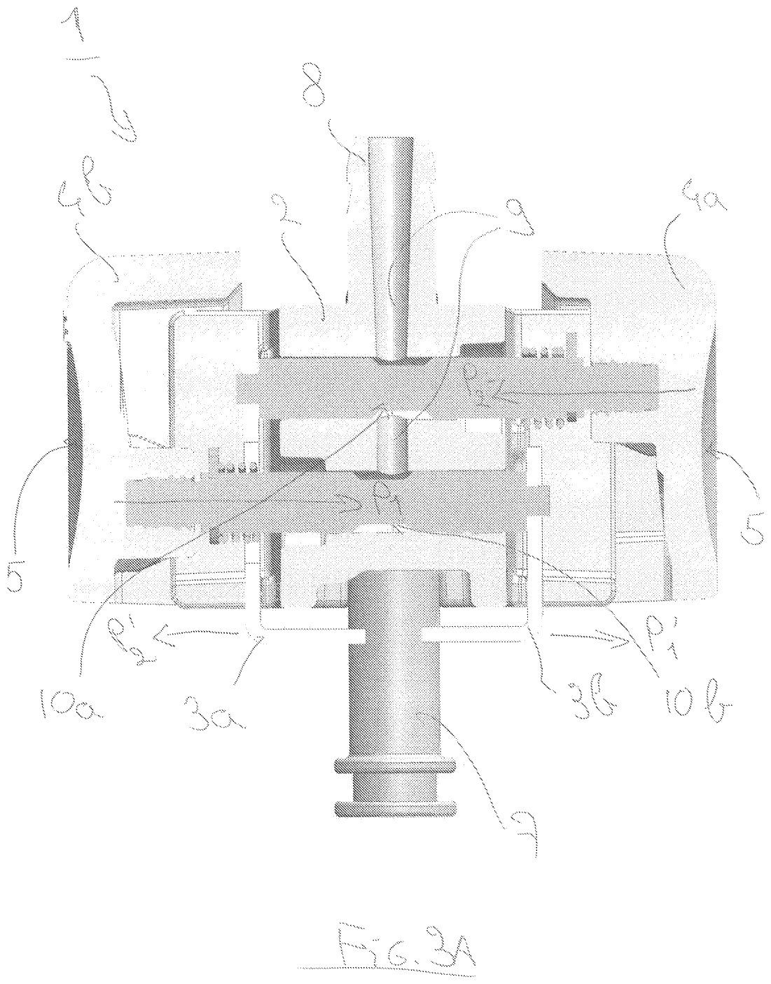

FIG. 2 a front view of the coupling device according to the present invention;

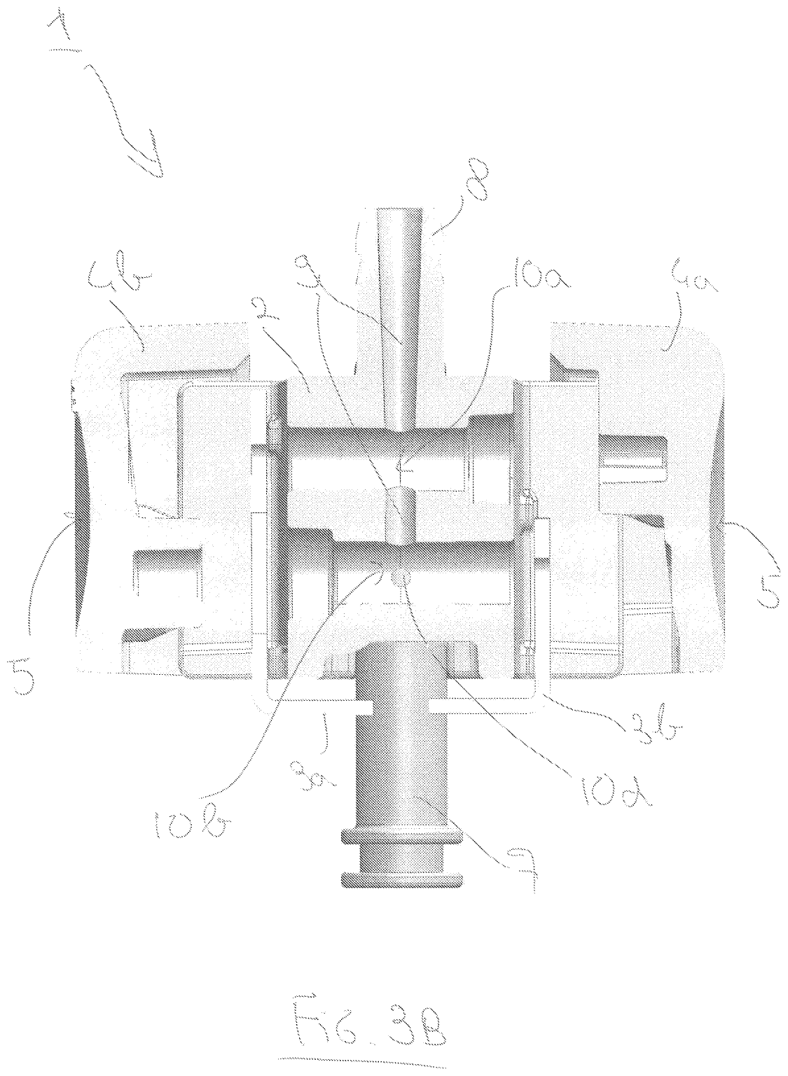

FIG. 3A and 3B a cross-sectional view of the coupling device according to the present invention.

FIGS. 1A and 1B shows a perspective view of the coupling device 1 according to the present invention. A device 1 is shown comprising a base part 2 and two cooperating engaging elements 3a, 3b. The device 1 further comprises grippers 4a, 4b which grippers 4a, 4b are connected to the cooperating engaging elements 3a, 3b. Both grippers 4a, 4b are provided with a fingertip-recess 5 (only the fingertip-recess 5 of gripper 4a is shown). The device 1 shown in FIGS. 1A and 1B further comprises a flush connector 8. The flush connector 8 is in direct communication with opening 10c (shown in FIG. 1A) debouching into a recess 6 provided in the base part 2 for receiving inserts 7 as shown in FIG. 1B. It is further noted that the two cooperating engaging elements 3a, 3b are mirrored with respect to each other. It is further noted that the contours of the two cooperating engaging elements 3a, 3b are chosen such that the contours of the two cooperating engaging elements 3a, 3b partially corresponds to the contours of at least one part of the medical instrument (not shown) to be received by the device 1.

FIG. 2 shows a front view of the coupling device 1 shown in FIGS. 1A and 1B, the device 1 shown in FIG. 2 is provided with one insert 7. The other insert 7 as shown in FIG. 1B is not shown in FIG. 2. Consequently, the recess 6 provided in the base part 2 is visualized, wherein the recess 6 is further provided with an opening 10c for flushing the recess 6 and/or an insert 7 (not shown) received by the recess 6. FIG. 2 thriller shows arrows P₁, P₂, representing a pressure exerted onto the grippers 4a, 4b in order to move the two cooperating engaging elements 3a, 3b in the direction indicated by arrows P₁′, P₂′. The two cooperating engaging elements 3a, 3b as shown in FIG. 2 are situated in the clamping position. By exerting a pressure in gripper 4a in the direction of arrow P2, the corresponding engaging element 3a will move into the direction indicated by arrow P₂′. Also, by exerting a pressure in gripper 4b in the direction of arrow P₁, the corresponding engaging element 3a will move into the direction indicated by arrow P₁′. Thus, in case the device 1 is squeezed by a user (not shown), i.e. narrowing the relative distance between both grippers 4a, 4b, the relative distance between the two cooperating engaging elements 3a, 3b is increased in order to bring the device 1 into the receiving position. By releasing the pressure exerted on the grippers 4a, 4, the two cooperating engaging elements 3a, 3b will return in their initial position, i.e. the clamping position, as illustrated in FIG. 2.

FIG. 3A and 3B shows a cross-sectional view of the coupling device 1 shown in FIGS. 1A and 1B. In FIG. 3A the connection between the gripper 4a and the engaging element 3a, as well as the connection between the gripper 4b and the engaging element 3b is visualized. Furthermore, the internal channel 9 is visualized comprising openings 10a, 10b for flushing the engaging elements 3a, 3b. In FIG. 3B the openings 10a, 10b are also shown including opening 10d debouching into a recess (not shown) provided with the recess 7.

1. Coupling device for a medical instrument, such as an endoscope, comprising:
a base part configured for cooperation with at least a part of the medical instrument; and
two cooperating engaging elements connected to the base part, wherein the cooperating engaging elements being movable between a first position and a second position, wherein:
the first position comprises a clamping position, wherein the engaging elements are located at a distance from each other such that the engaging elements clampingly engage at least one part of the medical instrument received by the device; and
the second position comprises a receiving position, wherein the engaging elements are located at a greater distance from each other relative to the first position such that the device is capable of receiving as well as releasing the at least one part of the medical instrument, wherein each of the cooperating engaging elements being linearly translationally movable between the clamping position and the receiving position, and wherein the translations of the cooperating engaging elements are located on a coinciding line.

2. Device according to claim 1, wherein the two cooperating engaging elements are substantially conformed, preferably mirrored with respect to one another.

3. Device according to claim 1, wherein the two cooperating engaging elements are L-shaped in cross-section, wherein the L-shape of the one engaging element is mirrored with respect to the L-shape of the other engaging element such that, in the clamping position, both engaging elements describe an interrupted U-shape in cross-section.

4. Device according to claim 1, wherein the two cooperating engaging elements describe at least a part of the contours of at least one part of the medical instrument to be received by the device.

5. Device according to claim 1, wherein the device is configured such that the engaging elements are under pretension in the receiving position.

6. Device according to claim 1, wherein each of the two cooperating engaging elements is connected to a grip for translationally moving the engaging element by a user.

7. Device according to claim 6, wherein the grips connected to the two cooperating engaging elements being linearly translationally movable between a first grip position and a second grip position, and wherein the translations of the grips are located on a coinciding line.

8. Device according to claim 6, wherein the grips connected to the two cooperating engaging elements in the first grip position are located at a greater distance from each other relative to the second grip position.

9. Device according to claim 6, wherein the first grip position corresponds to the clamping position of the engaging elements connected to the grips, and the second grip position corresponds to the receiving position of the engaging elements connected to the grips.

10. Device according to claim 6, wherein each of the grips connected to the cooperating engaging elements is provided with at least one fingertip-recess for translationally moving the grip with a fingertip of a user.

11. Device according to claim 6, wherein the device is configured such that the grips connected to the cooperating engaging elements are under pretension in the second grip position.

12. Device according to claim 6, wherein the base part comprises at least one insert, said insert is configured to cooperate with an at least one part of the medical instrument to be received by the device.

13. Device according to claim 6, wherein the base part comprises at least one recess for receiving an insert, said insert is configured to cooperate with an at least one part of the medical instrument to be received by the device.

14. Device according to claim 13, wherein the geometry of the at least one recess is chosen such that the insert can be received only by the base part in a predefined specific orientation.

15. Device according to claim 13, wherein the base part comprises at least two recesses for receiving inserts, wherein the geometry of the at least two recesses is chosen such that they are different with respect to each other.

16. Device according to claim 1, wherein the base part comprises a flush connector connectable to a flushing device, and wherein:
the flush connector is in direct communication with a channel provided in the base part; and
the channel comprises at least one opening for flushing one or more components of the device connected to the base part,

17. Device according to claim 16, wherein the channel comprises at least one opening for flushing the engaging elements.

18. (canceled)

19. (canceled)

20. Device according to claim 16, wherein the flushing device is part of a cleaning and disinfection machine or drying cabinet for medical instruments, such as endoscopes.

21. (canceled)

22. Method for coupling a device with a medical instrument, comprising the steps of;
a) providing a coupling device comprising:
a base part configured for cooperation with at least a part of the medical instrument, and two cooperating engaging elements connected to the base part, wherein the cooperating engaging elements being movable between a clamping position and a receiving position;
b) providing a medical device to be coupled with the medical instrument of step a);
c) bringing the engaging elements of the device provided in step a) in the receiving position;
d) receiving at least one part of the medical instrument provided in step b) by the device provided in step a); and
e) bringing the engaging elements of the device provided in step a) in the clamping position, wherein in steps c) and e) the engaging elements linearly translationally move between the receiving position and the clamping position, and wherein the translations of the cooperating engaging elements are located on a coinciding line.

23. Method according to claim 2, wherein the receiving position of the engaging elements is maintained by exerting pressure on the engaging elements, wherein the direction of the pressure exerted on the engaging elements corresponds to the direction of the translational movement of the engaging elements from the clamping position to the receiving position.

24. (canceled)

25. (canceled)

26. (canceled)

27. (canceled)
